(19) United States
(12) Patent Application Publication
(10) Pub. No.: US 2024/0058300 A1
(43) Pub. Date: Feb. 22, 2024

(54) TOCOTRIENOL COMPOSITIONS AND METHODS TO TREAT NON-ALCOHOLIC STEATOHEPATITIS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Savita KHANNA, Indianapolis, IN (US); Nandini GHOSH, Indianapolis, IN (US)

(21) Appl. No.: 18/260,115

(22) PCT Filed: Jan. 6, 2022
(86) PCT No.: PCT/US2022/011382

§ 371 Date: Jun. 30, 2023

Related U.S. Application Data
(60) Provisional application No. 63/134,552, filed on Jan. 6, 2021.

(51) Int. Cl. A61K 31/355; A61P 1/16

(52) U.S. Cl. CPC A61K 31/355; A61P 1/16

(57) ABSTRACT

This invention is directed to pharmaceutical compositions comprising tocotrienol and the use of such compositions to treat non-alcoholic steatohepatitis.

### CROSS REFERENCE TO RELATED APPLICATIONS

This application claims priority to the following: U.S. Provisional Patent Application No. 63/134,552 filed on Jan. 6, 2021, the disclosure of which is expressly incorporated herein.

### BACKGROUND OF THE DISCLOSURE

“Non-alcoholic fatty liver disease (NAFLD)” refers to a group of conditions where there is accumulation of excess fat in the liver of people who drink little or no alcohol. NAFLD comprises a wide spectrum of liver damage, ranging from simple macrovesicular steatosis to steatohepatitis, advanced fibrosis, and cirrhosis. The most common form of NAFLD is a non-serious condition called fatty liver. In fatty liver, fat accumulates in the liver cells. Although having fat in the liver is not normal, by itself it probably does not damage the liver. The majority of individuals with NAFLD have no symptoms and a normal examination. Children may exhibit symptoms such as abdominal pain that may be in the center or the right upper part of the abdomen, and sometimes fatigue. The liver might be slightly enlarged and some children may have patchy, dark discoloration of the skin present (acanthosis nigricans) most commonly over the neck and the under arm area. Symptoms can also include weight loss. Non-alcoholic fatty liver disease is one of the causes of fatty liver, occurring when fat is deposited (steatosis) in the liver due to causes other than excessive alcohol use. NAFLD is the most common liver disorder in developed countries. NAFLD is related to insulin resistance and the metabolic syndrome and is associated with diabetes mellitus type 2 and hyperlipidemia and obesity. NAFLD affects 30% of the world population and about 80% of obese people.

Non-alcoholic steatohepatitis (NASH) is a condition representing the most extreme form of NAFLD, and is regarded as a major cause of cirrhosis of the liver of unknown cause and can lead to the development of end stage liver disease and to primary liver cancer. About 2-8% of the world population suffers from NASH. Most patients with NAFLD will not develop NASH. NASH is a different clinical phenotype that is associated with unique genetic, metabolomic, lipidomic, and proteomic characteristics all of which differentiate patients that suffer from NASH from those with NAFLD. The only way to diagnose NASH is by a liver biopsy that differentiate patients with NASH from those with NAFLD. In many patients, nonalcoholic fatty liver disease is associated with metabolic risk factors such as obesity, diabetes mellitus, and dyslipidemia. Nonalcoholic fatty liver disease is histologically further categorized into nonalcoholic fatty liver (NAFL) and nonalcoholic steatohepatitis. Nonalcoholic fatty liver is defined as the presence of hepatic steatosis with no evidence of hepatocellular injury in the form of ballooning of the hepatocytes.

An ongoing, urgent need exists for new therapeutic interventions for NASH. The present invention addresses this need.

### SUMMARY

In one aspect, the disclosure relates to a method to treat non-alcoholic steatohepatitis (NASH) in a human subject in need thereof by administering to the subject a pharmaceutical composition comprising a therapeutically effective amount of a tocotrienol and an acceptable carrier. As used herein tocotrienol refers to compounds having the general structure of

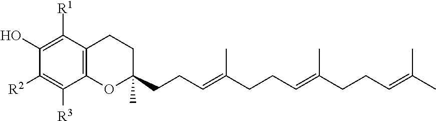

wherein R¹, R², and R³ are independently H or CH₃. In one embodiment R¹, R², and R³ are each CH₃ (α-tocotrienol). In one embodiment R¹, and R² are each CH₃ and R³ is H (β-tocotrienol). In one embodiment R¹ is H and R² and R³ is CH₃ (γ-tocotrienol). In one embodiment R¹, and R² are H and R³ is CH₃ (δ-tocotrienol).

In one aspect, the disclosure relates to a method of treating liver inflammation in a subject in need thereof by administering to the subject a pharmaceutical composition comprising a therapeutically effective amount of a tocotrienol and an acceptable carrier.

In one aspect, the disclosure relates to a method of treating liver fibrosis in a subject in need thereof by administering to the subject a pharmaceutical composition comprising a therapeutically effective amount of a tocotrienol and an acceptable carrier.

In one aspect, the disclosure relates to a method to increase liver stem cells (oval cells) in a subject in need thereof by administering to the subject a pharmaceutical composition comprising a therapeutically effective amount of a tocotrienol and an acceptable carrier.

In one aspect, the disclosure relates to a method of increasing the number of liver hepatocytes derived from liver stem cells (oval cells) in a subject in need thereof by administering to the subject a pharmaceutical composition comprising a therapeutically effective amount of a tocotrienol and an acceptable carrier.

In one aspect, the disclosure relates to a method to reduce lipid accumulation in the serum of a subject in need thereof by administering to the subject a pharmaceutical composition comprising a therapeutically effective amount of a tocotrienol and an acceptable carrier.

In one aspect, the disclosure relates to a method of reducing lipid accumulation in the liver of a subject in need thereof by administering to the subject a pharmaceutical composition comprising a therapeutically effective amount of a tocotrienol and an acceptable carrier.

In one aspect, the disclosure relates to a package comprising: (a) a pharmaceutical composition comprising a therapeutically effective amount of a tocotrienol and a pharmaceutically acceptable carrier; and, (b) instructions for use of the pharmaceutical composition to treat a human patient suffering from nonalcoholic steatohepatitis (NASH).

Additional embodiments, features, and advantages of the disclosure will be apparent from the following detailed description and through practice of the disclosure.

### BRIEF DESCRIPTION OF THE DRAWINGS

FIG. 1: NAFLD Activity Score (NAS) was developed as a tool to measure changes in NAFLD during therapeutic trials. The NAS can range from 0 to 8 and is calculated by the sum of scores of steatosis (0-3), lobular inflammation (0-3) and hepatocyte ballooning (0-2). In patients with NAFLD, NAS score of ≥5 strongly correlated with a diagnosis of “definite NASH” whereas NAS ≤3 correlated with a diagnosis of “not NASH”. FIG. 1 presents the results of a study of biopsies from livers of mice fed high fat diet (HFD) and supplemented with either placebo (corn oil) or tocotrienol (TCTRF). The histologically-based NAS score was derived from IU pathologist blinded. Data presented as mean±SEM (n=6).

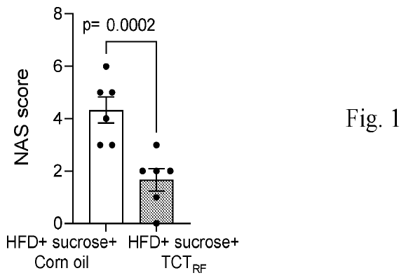

### DETAILED DESCRIPTION

### DEFINITIONS

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes alleviation of the symptoms associated with a specific disorder or condition and/or preventing or eliminating said symptoms. “Inhibition” of disease progression or disease complication in a subject means preventing or reducing the disease progression and/or disease complication in the subject.

As used herein an “effective” amount or a “therapeutically effective amount” of tocotrienol refers to a nontoxic but sufficient amount of tocotrienol to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring nucleic acid present in a living animal is not isolated, but the same nucleic acid, separated from some or all of the coexisting materials in the natural system, is isolated.

As used herein the term “patient” without further designation is intended to encompass any warm blooded vertebrate domesticated animal (including for example, but not limited to livestock, horses, mice, cats, dogs and other pets) and humans receiving a therapeutic treatment whether or not under the supervision of a physician.

“Administering” to a subject/patient means the giving of, dispensing of, or application of tocotrienols to a subject to relieve or cure a pathological condition. Oral administration is an example of administration used in the instant methods.

A subject at “baseline” is as subject before administration of a tocotrienol in a therapy.

“Concurrent administration,” “administration in combination,” “simultaneous administration,” or “administered simultaneously” mean that the tocotrienol compositions are administered at the same point in time or immediately following one another. In the latter case, the two compositions are administered at times sufficiently close that the results observed are indistinguishable from those achieved when the compositions are administered at the same point in time.

“Controlled-release” or “controlled-release delivery” refers to release or administration of a tocotrienol composition from a given dosage form in a controlled fashion to achieve the desired pharmacokinetic profile in vivo. An aspect of “controlled” delivery is the ability to manipulate the formulation and/or dosage form to establish the desired kinetics of tocotrienol release.

“Delayed release pharmaceutical composition” refers to a pharmaceutical composition comprising a tocotrienol where release of a tocotrienol occurs after passage of the pharmaceutical composition through the stomach. Preferably, the pharmaceutical composition is enterically coated. Preferably, the enteric coating imparts a delay in the release in the tocotrienol following oral administration of the pharmaceutical composition such that release of tocotrienol occurs after passage of the composition through the stomach. Optionally, the release of tocotrienol occurs after at least about 1 hour, at least about 2 hours, or at least about 3 hours after passage of the composition through the stomach. Alternatively, the release of tocotrienol occurs about 1 to about 3 hours or about 2 to about 3 hours after passage of the composition through the stomach. More preferably, the release of tocotrienol occurs about 5 to about 6 hours after ingestion.

“Non-alcoholic fatty liver disease (NAFLD)” refers to a group of conditions where there is accumulation of excess fat in the liver of people who drink little or no alcohol. NAFLD comprises a wide spectrum of liver damage, ranging from simple macrovesicular steatosis to steatohepatitis, advanced fibrosis, and cirrhosis.

“Nonalcoholic steatohepatitis (NASH)” is defined as the presence of hepatic steatosis and inflammation with hepatocyte injury (ballooning) with or without fibrosis. Nonalcoholic steatohepatitis can progress to cirrhosis, liver failure, and rarely liver cancer. Non-alcoholic fatty liver disease, comprising non-alcoholic fatty liver and NASH, is a multi-system disease with hepatic and extrahepatic manifestations, such as type 2 diabetes mellitus and cardiovascular disease; it affects 30% of the adult population and at least 10% of children. Non-alcoholic steatohepatitis related liver transplantations are predicted to eclipse other indications over the next decade, and both NAFLD and NASH have emerged as the dominant cause of hepatocellular carcinoma (HCC), the only cancer with rising incidence and third leading cause of cancer mortality. Besides fatty liver, NAFLD also encompasses non-alcoholic steatohepatitis and non-alcoholic fatty liver disease-associated cirrhosis.

“Obesity” refers to a chronic, relapsing health risk defined by excess body fat. The pathogenesis of obesity involves the interaction of genetic, environmental, and behavioral factors. Total body fat can be accurately measured using hydrodensitometry and dual-energy x-ray absorptiometry (DEXA). Because body mass index (BMI), expressed as kilograms of weight divided by height in meters squared (kg/m2), is simple and inexpensive to calculate, and correlates strongly with total body fat in non-elderly adults, it is commonly used as a surrogate for total body fat.

“Periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

“Subject” can refer to living organisms such as mammals, including, but not limited to humans, livestock, dogs, cats, and other mammals. Administration of the tocotrienol can be carried out at dosages and for periods of time effective for treatment of a subject. In some embodiments, the subject is a human. In some embodiments, the pharmacokinetic profiles of the systems of the present invention are similar for male and female subjects.

A “prophylactically effective amount” of a tocotrienol refers to an amount that is effective to prevent an unwanted physiological condition. Therapeutically effective and prophylactically effective amounts of a given therapeutic tocotrienol will typically vary with respect to factors such as the type and severity of the disorder or disease being treated and the age, gender, and weight of the subject. “Therapeutically effective amount” can also refer to an amount of a therapeutic tocotrienol, or a rate of delivery of a therapeutic tocotrienol (e.g., amount over time), effective to facilitate a desired therapeutic effect. The precise desired therapeutic effect will vary according to the condition to be treated, the tolerance of the subject, the tocotrienol and/or tocotrienol formulation to be administered (e.g., the potency of the therapeutic tocotrienol), the concentration of tocotrienol in the formulation, and the like), and a variety of other factors that are appreciated by those of ordinary skill in the art.

The natural vitamin E family is composed of eight members, equally divided into two classes; tocopherols (TCP) and tocotrienols (TCT or TE). TCPs are characterized by a saturated phytyl side chain with three chiral carbons whereas TCTs possess a famesyl side chain with double bonds at carbons 3, 7, and 11. Within each class, isomers are differentiated by α, β, γ, and δ according to the position and degree of methylation on the chromanol head. TCPs represent the primary form of vitamin E in green leafy vegetables, while TCTs are found in highest concentration in seeds of monocotyledons that include the wheat, rice, barley, and palm. In some embodiments, provided are such methods, where the tocotrienol composition is Tocovid SupraBio®. Tocovid SupraBio is a soft gel capsule that contains Tocomin, a natural extract of palm phytonutrients consisting of mixed-tocotrienols, tocopherols, mixed carotenoids, phytosterols and squalene. Mixed-tocotrienols are one of the key ingredients in Tocovid SupraBio and they are a more potent form of Vitamin E compared with tocopherol.

“Type II Diabetes Mellitus (T2DM)” refers to a syndrome characterized by hyperglycemia resulting from absolute or relative impairment in insulin secretion and/or insulin action. It is usually the type of diabetes diagnosed in patients at least 30 years old, but also occurs in children and adolescents. Symptoms include polyuria (dilute urine), polydipsia (extreme thirst), polyphagia (extreme hunger), weight loss, blurred vision, lower extremity paresthesias, or yeast infections, particularly balanitis in men. Genetic factors are the major determinants for T2DM. The presence of type 2 diabetes and other conditions associated with insulin resistance, such as polycystic ovarian syndrome, are known risk factors for the development of fatty liver and NASH.

“A patient afflicted with” or “a patient suffering from” NAFLD or NASH mean a subject who has been clinically diagnosed to have NAFLD or NASH, respectively.

### EMBODIMENTS

In accordance with one embodiment of the present disclosure a method of treating non-alcoholic steatohepatitis (NASH) in a human subject in need thereof, is provided, wherein the subject is administered a pharmaceutical composition comprising a therapeutically effective amount of a tocotrienol and an acceptable carrier. In one embodiment the pharmaceutical composition comprises 2, 3 or 4 tocotrienols selected from the group consisting of α-tocotrienol, β-tocotrienol, γ-tocotrienol and δ-tocotrienol.

In one embodiment a pharmaceutical tocotrienol composition is provided that comprises approximately 6-12% alpha-tocotrienol, approximately 0.7-1.1% beta-tocotrienol, approximately 8-16% gamma-tocotrienol, and approximately 3-7% delta tocotrienol with the remainder being non-active agents. In some embodiments, the tocotrienol composition comprises approximately 7.5% alpha-tocotrienol, approximately 0.9% beta-tocotrienol, approximately 11% gamma-tocotrienol, approximately 4.5% delta tocotrienol, and approximately 6% alpha tocopherol. In some embodiments, the tocotrienol composition comprises 6-12% alpha-tocotrienol, 0.7-1.1% beta-tocotrienol, 8-16% gamma-tocotrienol, 3-7% delta tocotrienol, and 4-10% alpha tocopherol. In some embodiments, the tocotrienol composition comprises 7.5% alpha-tocotrienol, 0.9% beta-tocotrienol, 11% gamma-tocotrienol, 4.5% delta tocotrienol, and 6% alpha tocopherol. Also provided are such methods, where the tocotrienol composition administered comprises tocopherol, by weight percent total, less than a percent selected from the group consisting of: 50%; 40%; 30%; 20%; 15%; 10%; 5%; and 1%.

In some embodiments, the tocotrienol composition in 1 ml of solution comprises: Tocomin 50%—200 mg; Soya Oil—305.4 mg; Labrasol—50 mg; and Cremophor—50 mg. Tocomin 50% typically comprises: d-Alpha-Tocotrienol: 61.52 mg {6.15% (w/v) of total capsule}; d-Gamma-Tocotrienol: 112.80 mg {11.28% (w/v) of total capsule}; d-Delta-Tocotrienol: 25.68 mg {2.57% (w/v) of total capsule}; d-Alpha-Tocopherol 91.60 IU; Plant Squalene 51.28 mg; Phytosterol Complex 20.48 mg; and Phytocarotenoid Complex 360.00 μg. In some embodiments, a single tocotrienol compound is administered. In some other embodiments, a tocotrienol rich fraction (TRF) is administered. TRF is an oily mixture of tocopherols and tocotrienols, in which tocotrienols constitutes 70-80% of the blend. The fundamental structural difference between the two groups is the phytyl chain, which is unsaturated in tocotrienols and saturated in tocopherols. The isoforms of tocopherols and tocotrienols differ from each other by the degree of methylation of the chromane ring. TRF is obtained from commercially available Tocovid Suprabio capsules. In some embodiments, each capsule contains Tocomin 50% that typically provides d-Alpha-Tocotrienol 61.52 mg, d-Gamma-Tocotrienol 112.80 mg, d-Delta-Tocotrienol 25.68 mg, d-Alpha-Tocopherol 91.60 IU, Plant Squalene 51.28 mg, Phytosterol Complex 20.48 mg, Phytocarotenoid Complex 360.00 μg. In some embodiments, each capsule contains 144.86 mM of d-Alpha-Tocotrienol.

While the tocotrienols for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In an embodiment, pharmaceutical compositions are provided comprising the active compounds or pharmaceutically acceptable salts or derivatives thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical compositions of the present disclosure may be administered by any convenient route, that suits the desired therapy. Preferred routes of administration include oral administration, in particular in tablet, in capsule, in dragée, in powder, or in liquid form, intranasal administration, intradermal administration, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

The compositions of the present disclosure may be formulated for simultaneous, separate or sequential administration, with at least a pharmaceutically acceptable carrier, additive, adjuvant or vehicle. This has the implication that the combination of the two active compounds may be administered:

as a combination that is being part of the same medicament formulation, the two active compounds being then administered always simultaneously; as a combination of two units, each with one of the active substances giving rise to the possibility of simultaneous, sequential or separate administration.

Further details on techniques for formulation and administration may be found in the latest edition of Remington's Pharmaceutical Sciences (Mack Publishing Co., Easton, Pa.).

Aspects of the present disclosure can be described as embodiments in any of the following enumerated clauses. It will be understood that any of the described embodiments can be used in connection with any other described embodiments to the extent that the embodiments do not contradict one another.

Clause 1. A method of reducing lipid accumulation in serum and liver tissues of patients in need thereof, wherein the method comprises the steps of:
administering to the subject a pharmaceutical composition comprising a therapeutically effective amount of a tocotrienol and an acceptable carrier, optionally wherein said patient has non-alcoholic steatohepatitis (NASH) and the administration of tocotrienol is used to treat non-alcoholic steatohepatitis (NASH) resulting in reduced inflammation, reduced liver fibrosis or reduced steatosis in liver tissue.

Clause 2. A method according to the preceding clause, where the human subject is also suffering from type II diabetes mellitus (T2DM), type I diabetes mellitus (T1DM), pre-diabetes or insulin resistance or obesity.

Clause 3. A method according to any of the preceding clauses, where the human patient has evidence of liver fibrosis, inflammation or steatohepatitis.

Clause 4. A method according to any of the preceding clauses, where the human patient does not have evidence of liver fibrosis.

Clause 5. A method according to any of the preceding clauses, where the pharmaceutical composition is a delayed release pharmaceutical composition.

Clause 6. A method according to any of the preceding clauses, where the pharmaceutical composition is a controlled release pharmaceutical composition.

Clause 7. A method according to any of the preceding clauses, where the composition is administered orally to the subject.

Clause 8. A method according to any of the preceding clauses, where the composition is administered periodically to the subject.

Clause 9. A method according to any of the preceding clauses, where the pharmaceutical composition is administered daily or twice daily.

Clause 10. A method according to any of the preceding clauses, where the pharmaceutical composition is administered twice per day.

Clause 11. A method according to any of the preceding clauses, where the pharmaceutical composition is administered less often than once daily.

Clause 12. A method according to any of the preceding clauses, where the pharmaceutical composition is administered to a patient who is contemporaneously receiving, or who has previously received, NASH therapy.

Clause 13. A method according to any of the preceding clauses, where the pharmaceutical composition is administered to a patient who is contemporaneously receiving any type of treatment for NAFLD, NASH, insulin resistance, or diabetes.

Clause 14. A method according to any of the preceding clauses, where the pharmaceutical composition is administered to a patient receiving NAFLD or NASH therapy selected from the group consisting of lipid-lowering medications, insulin-sensitizing medications, anti-oxidant medications, anti-apoptotic medications, and anti-cytokine medications.

Clause 15. A method according to any of the preceding clauses, where the pharmaceutical composition is administered to a patient who is contemporaneously receiving, or who has previously received T2DM therapy.

Clause 16. A method according to any of the preceding clauses, where the amount of tocotrienol administered to the patient per day is adjusted over time based on the patient's tolerability.

Clause 17. A method according to any of the preceding clauses, where administering the pharmaceutical composition results in improvement of a symptom of NAFLD, NASH, or T2DM relative to baseline after 1. 2, 3, 4, 5, 6, 7, 8, 9, 10, 11, 12, 13, 14, 15, 16, 17, 18, 19, 20, 21, 22, 23, or 24 weeks of administration.

Clause 18. A method according to any of the preceding clauses, where treatment results in the improvement of a symptom of NAFLD or NASH, selected from the group consisting of weight loss, fatigue, enlarged liver, or skin discoloration.

Clause 19. A method according to any of the preceding clauses, where treatment results in the improvement of a symptom of T2DM, selected from the group consisting of polyuria, polydipsia, polyphagia, weight loss, blurred vision, lower extremity paresthesias, or a yeast infection.

Clause 20. A method according to any of the preceding clauses, where treatment results in an improvement in insulin resistance.

Clause 21. A method according to any of the preceding clauses, where treatment results in improvement of a symptom of pre-diabetes.

Clause 22. A method according to any of the preceding clauses, where administration of the pharmaceutical composition results in resolution of NASH without worsening of fibrosis.

Clause 23. A method according to any of the preceding clauses, where the administration of the pharmaceutical composition results in an improvement on liver histology in NASH subjects with fibrosis.

Clause 24. A method according to any of the preceding clauses, where the administration of the delayed release pharmaceutical composition results in an improvement in histologic features of NASH from baseline to end of treatment liver biopsy.

Clause 25. A method according to any of the preceding clauses, where the administration of the pharmaceutical composition results in an improvement in in NASH activity, where NASH activity is defined by change in standardized scoring of liver biopsies at baseline and at end of treatment.

Clause 26. A method according to any of the preceding clauses, where the patient is administered the delayed release pharmaceutical composition for ≤24 weeks.

Clause 27. A method according to any of the preceding clauses, where the patient is administered the delayed release pharmaceutical composition for ≥24 weeks.

Clause 28. The method according to any of the preceding clauses, where the patient is also being administered immune modulatory agents.

Clause 29. A method according to any of the preceding clauses, where the tocotrienol composition is a tocotrienol rich fraction.

Clause 30. A method according to any of the preceding clauses, where the tocotrienol composition comprises at least one tocotrienol selected from the group consisting of: alpha-tocotrienol, beta-tocotrienol, gamma-tocotrienol, and delta-tocotrienol.

Clause 31. A method according to any of the preceding clauses, where the tocotrienol composition further comprises alpha-tocopherol.

Clause 32. A method according to any of the preceding clauses, where the tocotrienol composition comprises tocopherol, by weight percent of total, less than a percentage selected from the group consisting of: 50%; 40%; 30%; 20%; 15%; 10%; 5%; and 1%.

Clause 33. A method according to any of the preceding clauses, where the tocotrienol composition comprises 17-34% alpha-tocotrienol, 2-4% beta-tocotrienol; 27-54% gamma-tocotrienol, 8-23% delta tocotrienol, and 14-32% alpha tocopherol, by weight of those five ingredients.

Clause 34. A method according to any of the preceding clauses, where the tocotrienol composition is derived from palm oil.

Clause 35. A method according to any of the preceding clauses, where release of the tocotrienol occurs (a) at least 1 hour after passage of the pharmaceutical composition through the stomach or (b) about 5 to about 6 hours after ingestion of the pharmaceutical composition by the patient.

Clause 36. A method to treat a liver inflammation in a subject in need thereof, the method comprising the steps of:
administering to the subject a pharmaceutical composition comprising a therapeutically effective amount of a tocotrienol and an acceptable carrier.

Clause 37. A method according to the preceding clause, where the tocotrienol is a tocotrienol rich fraction.

Clause 38. A method according to any of the preceding clauses, where release of therapeutically effective amount of tocotrienol occurs (a) at least 1 hour after passage of the pharmaceutical composition through the stomach or (b) about 5 to about 6 hours after ingestion of the pharmaceutical composition by the patient.

Clause 39. A method to treat a liver fibrosis in a subject in need thereof, the method comprising the steps of:
administering to the subject a pharmaceutical composition comprising a therapeutically effective amount of a tocotrienol and an acceptable carrier.

Clause 40. A method according to the preceding clause, where the tocotrienol is a tocotrienol rich fraction.

Clause 41. A method according to any of the preceding clauses, where release of therapeutically effective amount of tocotrienol occurs (a) at least 1 hour after passage of the pharmaceutical composition through the stomach or (b) about 5 to about 6 hours after ingestion of the pharmaceutical composition by the patient.

Clause 42. A method to increase liver stem cells (oval cells) in a subject in need thereof, the method comprising the steps of:
administering to the subject a pharmaceutical composition comprising a therapeutically effective amount of a tocotrienol and an acceptable carrier.

Clause 43. A method according to the preceding clause, where the tocotrienol is a tocotrienol rich fraction.

Clause 44. A method according to any of the preceding clauses, where release of therapeutically effective amount of tocotrienol occurs (a) at least 1 hour after passage of the pharmaceutical composition through the stomach or (b) about 5 to about 6 hours after ingestion of the pharmaceutical composition by the patient.

Clause 45. A method to increase liver hepatocytes derived from liver stem cells (oval cells) in a subject in need thereof, the method comprising the steps of:
administering to the subject a pharmaceutical composition comprising a therapeutically effective amount of a tocotrienol and an acceptable carrier.

Clause 46. A method according to the preceding clause 45, where the tocotrienol is a tocotrienol rich fraction.

Clause 47. A method according to any of preceding clauses 45 or 46, where release of therapeutically effective amount of tocotrienol occurs (a) at least 1 hour after passage of the pharmaceutical composition through the stomach or (b) about 5 to about 6 hours after ingestion of the pharmaceutical composition by the patient.

Clause 48. A method to reduce lipid accumulation in a serum in a subject in need thereof, the method comprising the steps of:
administering to the subject a pharmaceutical composition comprising a therapeutically effective amount of a tocotrienol and an acceptable carrier.

Clause 49. A method according to preceding clause 48, where the tocotrienol is a tocotrienol rich fraction.

Clause 50. A method according to any of preceding clauses 48 or 49, where release of therapeutically effective amount of tocotrienol occurs (a) at least 1 hour after passage of the pharmaceutical composition through the stomach or (b) about 5 to about 6 hours after ingestion of the pharmaceutical composition by the patient.

Clause 51. A method to reduce lipid accumulation in a liver in a subject in need thereof, the method comprising the steps of:
administering to the subject a pharmaceutical composition comprising a therapeutically effective amount of a tocotrienol and an acceptable carrier.

Clause 52. A method according to preceding clause 51, where the tocotrienol is a tocotrienol rich fraction.

Clause 53. A method according to any of preceding clauses 51 or 52, where release of therapeutically effective amount of tocotrienol occurs (a) at least 1 hour after passage of the pharmaceutical composition through the stomach or (b) about 5 to about 6 hours after ingestion of the pharmaceutical composition by the patient.

Clause 54. A package comprising:
(a) a pharmaceutical composition comprising a therapeutically effective amount of a tocotrienol and a pharmaceutically acceptable carrier; and,
(b) instructions for use of the pharmaceutical composition to treat a human patient suffering from nonalcoholic steatohepatitis (NASH).

Clause 55. The package of clause 54 wherein the pharmaceutical composition comprising a therapeutically effective amount of a tocotrienol consists of 1, 2, 3 or 4 tocotrienols as the sole active agent wherein the tocotrienol active component is selected from the group consisting of α-tocotrienol, β-tocotrienol, γ-tocotrienol and δ-tocotrienol.

Other variations or embodiments will be apparent to a person of ordinary skill in the art from the above-description. Thus, the foregoing embodiments are not to be construed as limiting the scope of the claimed invention. All references disclosed are expressly incorporated by reference in in their entirety.

### EXAMPLES

To investigate the protective mechanisms of tocotrienol rich fraction (TCTRF) supplementation against progression of Non-Alcoholic Steatohepatitis in murine liver tissue, C57bl/6 mice (at 8 weeks) were fed a High Fat Diet (HFD), with or without oral gavaging 5 days/week and body weight and serum liver analysis was conducted over a twelve week period with analysis of liver tissue and serum content at the end of twelve weeks.

1. Animal model to study NASH is established.

To set up the model, mice (n=20) fed High Fat Diet (HFD) for 12 weeks were not supplemented with Tocotrienol rich fraction (TRF or TCTRF). These mice liver showed steatosis, inflammation and fibrosis. Steatosis was determined by histology (quantified by Hematoxylin & Eosin (H & E) staining, and fat staining such as Nile Red staining). Inflammation was measured by the elevated expression of genes such as CD34 and TNFα. Fibrosis was determined by histology (Masson's Trichome staining) and collagen 1 and 3 staining.

2. Animal cohort (n=20) fed HFD and supplemented with TRF for 12 weeks is completed.

Tocotrienol supplemented mice liver histology showed improved steatosis (quantified by Hematoxylin & Eosin (H & E) staining, Nile Red staining & Osmium tetraoxide staining).

Analysis of inflammation and fibrosis of the TRF supplemented mice liver is completed. Inflammation markers such as TNFα, IL-10 and CD34 were analyzed. TRF reduced pro-inflammatory marker TNFα levels and increased anti-inflammatory marker IL-10 levels in liver of TRF supplemented mice. TRF reduces the fibrosis in mice livers as analyzed by Masson's Trichome staining, collagen 1 and 3 staining.

We found that TRF supplementation induces stem cells (oval cells) in liver. Oval cells are bipotent liver cells that can either transdifferentiate to cholangiocytes or hepatocytes. We observed that TRF induces oval cells to hepatocyte conversion on TRF supplemented mice liver. CK19, a marker of oval cell, was found to be induced in TRF gavaged mice liver as compared to the placebo supplemented mice.

To further investigate the induction of oval cells in TRF supplemented, we generated a transgenic mouse, CK19Rosa, where all the CK19 positive cells well fluoresce GFP. The CK19Rosa mice were fed HFD and supplemented with either placebo or TRF for 12 weeks. CK19Rosa mice supplemented with TRF showed higher percentage of GFP+ cells. We stained the mice liver with albumin and Hnfα, hepatocyte markers. TRF supplemented mice liver showed higher number of colocalized cells, GFP+Albumin+ cells. TRF supplemented mice liver also showed high higher number of colocalized cells, GFP+Hnfα+ cells. TRF supplemented mice liver also showed reduced expression of Notch1 indicating its role in oval cell-hepatocyte conversion.

Non-alcoholic steatosis (NASH) is characterized by steatosis (accumulation of fat), increased inflammation and fibrosis of the liver. Oral TRF treatment improves liver condition by:
1. reducing the liver injury enzymes—aspartate aminotransferase (AST) & alanine aminotransferase (ALT) in the serum.
2. reducing steatosis in liver tissue as analyzed by Hematoxylin & Eosin (H&E) and Nile Red staining.
3. reducing inflammation in liver as analyzed by CD34 expression and TNFα and IL-10 levels.
4. reducing fibrosis as analyzed by Masson's Trichome and collagen 1 and 3 staining of liver tissue.
5. by inducing stem cell niche of liver. TRF induces a particular liver stem cells known as oval cells (bipotent cells) to transdifferentiate to hepatocytes.
6. TRF induced oval cell to hepatocyte transdifferentiation is shown by CK19Rosa transgenic mice (CK19, a marker for ovals, are GFP positive in this mice strain).
7. TRF supplemented CK19Rosa mice showed increased GFP positive cells in their portal regions. Followed by staining with albumin, a marker for hepatocytes, the TRF supplemented mice showed higher percentage of co-localized GFP+ and albumin+ cells in their portal regions of the liver.
8. It was found that TRF induced oval cell to hepatocyte transdifferentiation is Notch1 dependent. TRF reduced Notch1 in liver to facilitate oval-hepatocyte transdifferentiation.

1. A method to treat non-alcoholic steatohepatitis (NASH) in a human subject in need thereof, the method comprising the steps of:
administering to the subject a pharmaceutical composition comprising a therapeutically effective amount of a tocotrienol and an acceptable carrier.

2. A method to treat a liver inflammation or liver fibrosis in a subject in need thereof, the method comprising the steps of:
administering to the subject a pharmaceutical composition comprising a therapeutically effective amount of a tocotrienol and an acceptable carrier.

3. (canceled)

4. A method of reducing lipid accumulation in serum and liver tissues of patients in need thereof, or increasing the number of liver stem cells (oval cells), or liver hepatocytes derived from liver stem cells, in a subject in need of such increased cells, the method comprising the steps of:
administering to the subject a pharmaceutical composition comprising a therapeutically effective amount of a tocotrienol and an acceptable carrier.

5. (canceled)

6. A kit for treating non-alcoholic steatohepatitis (NASH) in a human subject in accordance with claim 1, said kit comprising:
(a) a pharmaceutical composition comprising a therapeutically effective amount of a tocotrienol selected from the group consisting of α-tocotrienol, β-tocotrienol, γ-tocotrienol and δ-tocotrienol; and a pharmaceutically acceptable carrier; and,
(b) instructions for use of the pharmaceutical composition to treat a human patient suffering from nonalcoholic steatohepatitis (NASH).

7. The method of claim 1 wherein the tocotrienol is administered orally.

8. The method of claim 1 wherein the tocotrienol is administered as a pharmaceutical composition comprising a mixture of tocopherols and tocotrienols.

9. The method of claim 1 wherein the tocotrienol is administered as a pharmaceutical composition comprising a tocotrienol compound selected from the group consisting of α-tocotrienol, β-tocotrienol, γ-tocotrienol and δ-tocotrienol.

10. The method of claim 9 wherein the tocotrienol composition comprises 17-34% alpha-tocotrienol, 2-4% beta-tocotrienol; 27-54% gamma-tocotrienol, 8-23% delta tocotrienol, and 14-32% alpha tocopherol, by weight, of those five ingredients.

11. The method of claim 9 wherein the pharmaceutical composition comprises a single tocotrienol compound selected from the group consisting of α-tocotrienol, β-tocotrienol, γ-tocotrienol and δ-tocotrienol.

12. The method of claim 11 wherein the single tocotrienol compound is α-tocotrienol.

13. The method of claim 1 wherein said tocotrienol is administered in conjunction with lipid-lowering medications, insulin-sensitizing medications, anti-oxidant medications, anti-apoptotic medications, or anti-cytokine medications.

14. The method of claim 7 wherein the tocotrienol is formulated as a controlled release formulation where the release of a therapeutically effective amount of tocotrienol occurs (a) at least 1 hour after passage of the pharmaceutical composition through the stomach or (b) about 5 to about 6 hours after ingestion of the pharmaceutical composition by the patient.
